SEQUENCE LISTING (10 sequences)

SEQ ID NO: 1 - DNA, 22 bp, synthetic construct
  source 1..22: mol_type = other DNA
ggacaagtacctggagagcaga

SEQ ID NO: 2 - DNA, 22 bp, synthetic construct
  source 1..22: mol_type = other DNA
gggatgttctcactggctaacg

SEQ ID NO: 3 - DNA, 20 bp, synthetic construct
  source 1..20: mol_type = other DNA
aatagtgggcaactggaggg

SEQ ID NO: 4 - DNA, 20 bp, synthetic construct
  source 1..20: mol_type = other DNA
gaacagagcaggcaggtgaa

SEQ ID NO: 5 - DNA, 20 bp, synthetic construct
  source 1..20: mol_type = other DNA
tctgacaaaccttcatgtcc

SEQ ID NO: 6 - DNA, 22 bp, synthetic construct
  source 1..22: mol_type = other DNA
aaatagtgataccgtagatgcg

SEQ ID NO: 7 - DNA, 20 bp, synthetic construct
  source 1..20: mol_type = other DNA
tgaccttctctcctccatcc

SEQ ID NO: 8 - DNA, 20 bp, synthetic construct
  source 1..20: mol_type = other DNA
cttcctgggagtctcatcct

SEQ ID NO: 9 - DNA, 23 bp, synthetic construct
  source 1..23: mol_type = other DNA
ggagattactgccctggctccta

SEQ ID NO: 10 - DNA, 24 bp, synthetic construct
  source 1..24: mol_type = other DNA
gactcatcgtactcctgcttgctg

(19) United States
(12) Patent Application Publication
(10) Pub. No.: US 2025/0090558 A1
(43) Pub. Date: Mar. 20, 2025

(54) APPLICATION OF MILTEFOSINE IN PROMOTING BONE FORMATION AND PREVENTING-TREATING OSTEOPOROSIS

(71) Applicant: The First Affiliated Hospital of Zhejiang Chinese Medical University, Hangzhou (CN)

(72) Inventors: Hongting Jin, Hangzhou (CN); Qinwen Ge, Hangzhou (CN)

(21) Appl. No.: 18/779,102

(22) Filed: Jul. 22, 2024

(30) Foreign Application Priority Data
Sep. 18, 2023 (CN) 202311201886.1

(51) Int. Cl. A61K 31/661; A61P 19/10

(52) U.S. Cl. CPC A61K 31/661; A61P 19/10

(57) ABSTRACT

An application method of miltefosine includes promoting bone formation and preventing and treating osteoporosis, which significantly increases an expression of protein phosphatase magnesium-dependent 1A(PPM1A) in osteoblasts during the bone formation process, regulates the dephosphorylation of a core protein of Smad2 in the transforming growth factor-β (TGF-β) signaling pathway, thereby inhibiting the TGF-β signaling pathway and promoting the expression of osteogenic markers, enhancing the body's bone-forming capacity. The miltefosine as a PPM1A enzyme activity catalyst, which, when administered in vivo, can significantly accelerate bone repair and delay bone loss in postmenopausal osteoporosis. Its mechanism involves catalyzing the enzymatic activity of PPM1A in osteoblasts, promoting the dephosphorylation of the Smad2 protein, and inhibiting the activity of the TGF-β/Smad2 signaling pathway, thereby promoting bone formation, accelerating bone repair, and preventing bone loss. Therefore, miltefosine has a promising application prospect for clinical treatment in promoting osteogenesis and preventing and treating osteoporosis.

### TECHNICAL FIELD

The disclosure relates to the technical field of small molecular compounds, and particularly to an application of miltefosine (also referred to as hexadecylphosphocholine) in promoting bone formation and preventing-treating osteoporosis (OP).

### STATEMENT REGARDING SEQUENCE LISTING

The sequence listing associated with this application is provided in text format in lieu of a paper copy and is hereby incorporated by reference into the specification. The name of the XML file containing the sequence listing is 24068TBYX-USP1-SL.xml. The XML file is 10,035 bytes; is created on Jul. 8, 2024; and is being submitted electronically via patent center.

### BACKGROUND

Postmenopausal osteoporosis (PMOP) is a common bone disease caused by the deficiency of estrogen after menopause, characterized mainly by bone mass loss and bone microstructure damage. Statistics show that about 50% of postmenopausal women worldwide are affected by OP to varying degrees, and the incidence of fragility fractures caused by PMOP is as high as 40%. PMOP has become a global public health issue due to its high incidence and serious harm. PMOP is gradually becoming a major threat to the health of the postmenopausal women, and it is urgent to actively explore the pathogenesis of PMOP. However, there is still a lack of effective intervention measures to stop the progression of OP, mainly because the cellular and molecular mechanisms of bone formation in the body are not fully elucidated.

Existing studies indicate that the transforming growth factor-β (TGF-β) signaling pathway plays a crucial role in maintaining bone homeostasis and the development of osteoarthritis. An increasing number of studies show that the TGF-β signaling pathway is involved in the maintaining bone homeostasis of the body. Smad2/3 (i.e., Smad2 and Smad3) is considered one of the key components in the classical TGF-β signaling pathway. The core step of the TGF-β signaling pathway is the phosphorylation of the Smad2/3 induced by the TGF-β receptor I kinase (TGFβRI). Then, the phosphorylated Smad2/3 (p-Smad2/3) combines with Smad4 to form a complex that shuttles into the nucleus and participates in transcription.

Protein phosphatase magnesium-dependent 1A (PPM1A) is a member of the metal-dependent protein phosphatases (PPMs) family, capable of dephosphorylating phosphoserine residues and phosphothreonine residues. Notably, PPM1A has been identified as an effective protein phosphatase for a variety of substrates, with Smad2, a key regulatory factor in the classical TGF-β signaling pathway, being one of them. Considering that the TGF-β signaling pathway is one of the important pathways involved in the regulation of bone homeostasis and the pathogenesis of OP, PPM1A, as a phosphatase for p-Smad2, can participate in the regulation of the activity of the TGF-β signaling pathway. Therefore, the regulatory role of PPM1A on osteoblasts is clarified as being of significant importance for discovering new targets and related drugs for the prevention-treatment of OP.

Miltefosine (MF), on Mar. 19, 2014, was approved by the U.S. Food and Drug Administration (FDA) primarily for the treatment of three types of leishmaniasis (e.g., visceral leishmaniasis, cutaneous leishmaniasis, and mucocutaneous leishmaniasis), and has been used clinically for palliative treatment of several cancers that are difficult to treat with conventional therapies. Recently, MF has been reported as an enzyme activity catalyst for PPM1A, improving the disease progression of Alzheimer's. However, the therapeutic effect of MF on bone diseases has not yet been reported.

### SUMMARY

Aiming to address the shortcomings in related art, the purpose of the disclosure is to provide a new function for the drug of miltefosine, namely, the function of promoting bone formation and preventing-treating OP. The disclosure intends to promote bone formation by catalyzing the activity of PPM1A in osteoblasts, thereby inhibiting the excessive activation of the TGF-β signaling pathway in osteoblasts, and simultaneously provides an application of miltefosine in a preparation of drugs for prevention and treatment of OP.

The technical solutions of the disclosure are as follows.

Specifically, an application method of miltefosine includes: promoting bone formation and preventing and treating osteoporosis by using the miltefosine. The miltefosine is hexadecylphosphocholine and the miltefosine (C₂₁H₄₆NO₄P) is shown in a formula I, and the formula I is expressed as follows:

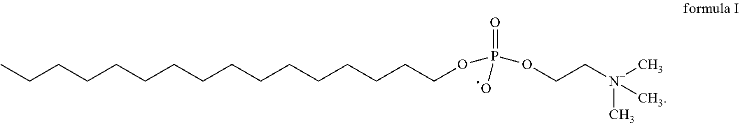

The miltefosine is used to enhance osteogenic differentiation of osteoblasts.

The miltefosine is used to increase a bone density of newly formed bone in a repair area.

The miltefosine is used to increase a bone volume fraction of the newly formed bone in the repair area.

The miltefosine is used to enhance a trabecular bone of the newly formed bone in the repair area.

The miltefosine is used to increase a bone density in a distal femur of a postmenopausal women.

The miltefosine is used to reduce a trabecular separation in the distal femur of the postmenopausal women.

The miltefosine is used to reduce bone loss in the distal femur of the postmenopausal women and to increase a number of the osteoblasts on a surface of the trabecular bone.

In an embodiment, the miltefosine is an osteogenic agent and is in a form of a solid powder.

In an embodiment, the miltefosine is a medication catalyzing an activity of protein phosphatase magnesium-dependent 1A (PPM1A) in the osteoblasts.

In an embodiment, the osteoporosis includes postmenopausal osteoporosis.

In an embodiment, an effective dose of the miltefosine for the promoting bone formation is in a range of 5-10 milligrams per kilogram (mg/kg) body weight.

In normal human osteoblasts, PPM1A is highly expressed. When human suffer from osteoporosis, the expression of PPM1A is downregulated. The miltefosine of the PPM1A enzyme activity catalyst promotes bone formation by regulating the TGF-β/Smad2 signaling pathway in osteoblasts.

Compared to the related art, the benefits of the disclosure are as follows.

The miltefosine can promote the differentiation of osteoblasts and accelerate the repair of bone defects, while also improving bone loss in postmenopausal osteoporosis. Therefore, the miltefosine has the potential for clinical intervention in the progression of bone defects and osteoporosis.

### BRIEF DESCRIPTION OF DRAWINGS

The attached drawings here are incorporated into the specification and form a part of the specification, illustrating embodiments in accordance with the disclosure and used together with the specification to explain the principles of the disclosure.

FIG. 1A illustrates a chemical structural formula diagram of miltefosine.

FIG. 1B illustrates cell viability diagram of primary osteoblasts under interventions of different concentrations of the miltefosine.

FIG. 1C illustrates an alkaline phosphatase (ALP) staining diagram of the osteoblasts under the interventions of different concentrations of the miltefosine.

FIG. 1D illustrates messenger ribonucleic acid (mRNA) expression levels of osteogenic genes of Sp7 transcription factor (Sp7), bone gamma-carboxyglutamate protein (Bglap), and alkaline phosphatase (Alpl) in the primary osteoblasts under the interventions of different concentrations of the miltefosine.

FIG. 1E illustrates a micro computed tomography (Micro-CT) scan diagram of bone defect repair in a mouse model treated with the miltefosine with a scale bar of 1 millimeter (mm).

FIG. 1F illustrates a quantitative analysis of bone microstructure of newly formed bone tissue in bone defect of the mouse model one week under the intervention of the miltefosine after operation.

FIG. 1G illustrates a safranin O/fast green staining (SO/FG) staining diagram of a repair site in the bone defect of the mouse model one week under the intervention of the miltefosine after operation, with a scale bar of 1 mm.

FIG. 1H illustrates a quantitative analysis diagram of the newly formed bone area (i.e., new bone area) at the repair site of the bone defect of the mouse model one week after operation.

FIG. 1I illustrates an immunohistochemical staining diagram of osteocalcin (OCN) at the bone repair site in the bone defect of the mouse model with a scale bar of 1 mm.

FIG. 1J illustrates a quantitative analysis diagram of the OCN immunohistochemical staining at the bone repair site in the bone defect of the mouse model.

FIG. 1K illustrates a representative Micro-CT scan diagram of the bone defect repair in a wild-type (Ppm1a^{+/+}) mice and a PPM1A knockout (Ppm1a^{-/-}) mice treated with the miltefosine with a scale bar of 50 micrometers (μm).

FIG. 1L illustrates a quantitative analysis of the bone microstructure of the newly formed bone tissue in the bone defect of the mouse model one week under interventions with different concentrations of the miltefosine after operation, with data represented as mean±standard deviation (* indicates P<0.05, and ** indicates P<0.0).

FIG. 2A illustrates a representative Micro-CT scan diagram of a distal femur in ovariectomy (OVX) model mice eight weeks after operation with an intervention of the miltefosine, with a scale bar of 1 mm.

FIG. 2B illustrates a quantitative analysis diagram of bone mineral density (BMD) in a trabecular bone of the distal femur in the OVX model mice eight weeks after operation with the intervention of the miltefosine.

FIG. 2C illustrates a quantitative analysis diagram of a structural model index (SMI) in the trabecular bone of the distal femur in the OVX model mice eight weeks after operation with the intervention of the miltefosine.

FIG. 2D illustrates a quantitative analysis diagram of a connectivity density (Conn.Dn) in the trabecular bone of the distal femur in the OVX model mice eight weeks after operation with the intervention of the miltefosine.

FIG. 2E illustrates a quantitative analysis diagram of a bone volume fraction (i.e., bone volume per tissue volume abbreviated as BV/TV) in the trabecular bone of the distal femur in the OVX model mice eight weeks after operation with the intervention of the miltefosine.

FIG. 2F illustrates a quantitative analysis diagram of a trabecular number (Tb.N) in the distal femur of the OVX model mice eight weeks after operation with the intervention of the miltefosine.

FIG. 2G illustrates a quantitative analysis diagram of a trabecular thickness (Tb.Th) in the distal femur of the OVX model mice eight weeks after operation with the intervention of the miltefosine.

FIG. 2H illustrates a quantitative analysis diagram of a trabecular separation (Tb.Sp) in the distal femur of the OVX model mice eight weeks after operation with the intervention of the miltefosine.

FIG. 2I illustrates a representative alizarin red and osteographin (ABH/OG) staining diagram in the distal femur of the OVX model mice eight weeks after operation through the intervention of the miltefosine, with a scale bar of 0.5 mm.

FIG. 2J illustrates a quantitative analysis diagram of a trabecular bone area fraction in the distal femur of the OVX model mice eight weeks after operation with the intervention of the miltefosine.

FIG. 2K illustrates a quantitative analysis diagram of a number of osteoblasts in the distal femur of the OVX model mice eight weeks after operation with the intervention of the miltefosine.

FIG. 2L illustrates a representative immunofluorescence diagram of OCN in the distal femur of the OVX model mice eight weeks after operation with the intervention of the miltefosine, with a scale bar of 50 km.

FIG. 2M illustrates a quantitative analysis diagram of an OCN expression in the distal femur of the OVX model mice eight weeks after operation with the intervention of the miltefosine.

FIG. 2N illustrates a representative immunohistochemical diagram of ALP in the distal femur of the OVX model mice eight weeks after operation with the intervention of the miltefosine.

FIG. 2O illustrates a quantitative analysis diagram of ALP expression in the distal femur of the OVX model mice eight weeks after operation with the intervention of the miltefosine.

FIG. 3A illustrates a representative tartrate resistant acid phosphatase (TRAP) staining diagram of the distal femur in the OVX model mice eight weeks after operation with the intervention of the miltefosine with a scale bar of 0.5 mm.

FIG. 3B illustrates a quantitative analysis diagram of a number of the osteoclasts in the distal femur of the OVX model mice eight weeks after operation with the intervention of the miltefosine.

FIG. 3C illustrates a representative immunofluorescence diagram of cathepsin K (CTSK) in the distal femur of the OVX model mice eight weeks after operation with the intervention of the miltefosine, with a scale bar of 50 m.

FIG. 3D illustrates a quantitative analysis diagram of a CTSK expression in the distal femur of the OVX model mice eight weeks after operation with the intervention of the miltefosine.

FIG. 3E illustrates a representative immunohistochemical diagram of fatty acid binding protein 4 (Fabp4) in the distal femur of the OVX model mice eight weeks after operation through the intervention of the miltefosine with a scale bar of 50 μm.

FIG. 3F illustrates a quantitative analysis diagram of a Fabp4 expression in the distal femur of the OVX model mice eight weeks after operation with the intervention of the miltefosine.

FIG. 3G illustrates a representative hematoxylin and eosin (HE) staining diagram of liver, heart, kidney, and spleen in the OVX model mice eight weeks after operation with the intervention of the miltefosine.

### DETAILED DESCRIPTION OF EMBODIMENTS

The following will provide a detailed explanation of embodiments, which are illustrated in the attached drawings. When the following description involves drawings, unless otherwise indicated, the same numbers in different drawings represent the same or similar elements. The embodiments described in the following exemplary embodiments do not represent all embodiments consistent with the disclosure.

The terms used in the disclosure are for the purpose of describing specific embodiments only and are not intended to limit the disclosure. The singular forms used in the disclosure and the claims are also intended to include the majority form, unless the context clearly indicates otherwise. It should also be understood that the term “and/or” used in the disclosure refers to and includes any or all possible combinations of one or more related listed items.

The disclosure will be further described in conjunction with specific embodiments, but the scope of protection of the disclosure is not limited to the embodiment.

### Embodiment 1

A study on a mechanism of miltefosine promoting bone formation

### 1.1 Method

### 1.1.1 Construction of Bone Defect of a Mouse Model

The 10-week-old mouse is anesthetized with 2% isoflurane gas. After adequate anesthesia, a right knee joint area of the mouse is shaved, and skin on the shaved right knee joint area is incised along a midline of the shaved right knee joint to expose a patellar ligament. A medial margin of the patellar ligament is cut with an ophthalmic scissor to expose a cavity of the right knee joint. The patella is placed in an external dislocation state, and a hole is drilled in an intercondylar region of the femur using a 26-gauge needle. A Kirschner wire with 0.6 mm diameter is inserted from a femoral condyle to a proximal femur. After removing the Kirschner wire, the dislocated patella is relocated, and the skin is closed by suturing. The mouse is euthanized 7 days post-surgery.

### 1.1.2 Animal Sample Processing and Section Preparation

The mouse is euthanized with carbon dioxide (CO₂) at one week of post-surgery, and animal samples from the knee joint areas are collected. The animal samples are then subjected to tissue dissection, and followed by placing the dissected tissue samples in 4% paraformaldehyde (PFA) for fixation at room temperature for 3 days. After the fixation, the fixed tissue samples are decalcified in a 14% Ethylene diamine tetra acetic acid (EDTA) solution for 2 weeks, with the decalcification solution being changed every other day. After the decalcification, the decalcified tissue samples are dehydrated using an automatic dehydrator. All tissue samples are embedded in paraffin to obtain paraffin blocks and then sectioned to obtain tissue sample sections, with a section thickness of 4 micrometers (m).

### 1.1.3 Imaging Observation

All the tissue sample sections are scanned using a high-resolution micro-CT (μ-CT) scanner with scanning parameters set to 45 kilovoltages (kV), 500 microamperes (μA), and an exposure time of 780 microseconds (ms). A region of interest (ROI) for bone repair is selected from the distal femur, and quantitative analysis of bone microstructural parameters such as bone mineral density (BMD), bone volume fraction (i.e., bone volume per tissue volume abbreviated as BV/TV), and trabecular number (Tb.N) is performed on the ROI.

### 1.1.4 Observation of Organizational Morphology

Before staining, the tissue sample sections are placed in an oven at 60° C. for 4 hours to enhance the adhesion of the tissues to the slides. Then, the tissue sample sections are taken out to stand for 15 minutes to return to room temperature. The tissue sample sections are then deparaffinized and rehydrated using xylene and a graded series of alcohol. Subsequently, the tissue sample sections are stained using a safranin-O/fast green (SO/FG) staining. A specific staining steps are as follows: first, the tissue sample sections are immersed in a 0.05% fast green solution for 3 minutes, then placed in a 3% glacial acetic acid solution for 10 seconds, followed by staining in a 2.5% safranin-O solution for 10 minutes. After the staining, the tissue sample sections are rinsed with distilled water three times, each for 3 minutes, and then dehydrated through a graded series of alcohol and cleared with xylene, and followed by sealing the tissue sample sections using a mounting medium.

### 1.1.5 Immunohistochemical Analysis

The tissue sample sections are taken to place in an oven at 60° C. to bake overnight. The next day, the tissue sample sections are taken out to stand for 15 minutes to return to room temperature, then the tissue sample sections are deparaffinized and rehydrated using xylene and a graded series of alcohol. A heat retrieval method (also referred to as heat fixation method) is employed by immersing the tissue sample sections in a sodium citrate buffer solution and placing the tissue sample sections in the sodium citrate buffer solution in an oven at 60° C. for 4 hours to complete antigen retrieval, thereby exposing antigen epitopes fully. The tissue sample sections are treated with a 0.3% Triton X-100 solution for permeabilization, then 100 μL of an endogenous peroxidase blocker are added into each tissue sample section to incubate at room temperature for 10 minutes. After the incubating, 100 μL of a normal goat serum working solution are added into each issue sample section for blocking, followed by incubating at room temperature for 15 minutes, and then the normal goat serum in each issue sample section is discarded. After the discarding, 100 μL of a primary antibody reagent are added into each issue sample section to incubate overnight at 4° C. The next day, the issue sample sections are taken out to return to room temperature, the primary antibody reagent in each issue sample section is washed off with phosphate buffer saline (PBS) buffer, and then 100 μL of biotin-labeled goat anti-rabbit IgG polymer, 100 μL of horseradish peroxidase-labeled streptavidin working solution are sequentially added into each issue sample section, and then an appropriate amount of freshly prepared diaminobenzidine (DAB) solution is added for a color development. After the color development is complete, the tissue sample sections are placed in a hematoxylin staining solution for 20 seconds and differentiated with 1% hydrochloric acid alcohol, and followed by rinsing with tap water to blue. Finally, the tissue sample sections are dehydrated through a graded series of alcohol and cleared with xylene, and the tissue sample sections are sealed with neutral resin.

### 1.1.6 Isolation and Culture of Primary Osteoblasts

After euthanizing 3-5-day-old mouse with CO₂, the euthanized mouse is soaked in 70% alcohol for disinfection for 15 minutes. The disinfected mouse is then carefully skinned from a top of the skull, and the skull tissue blocks are placed in an a-modified eagle medium (α-MEM) digestion culture medium containing 1 mg/mL collagenase-A and 1% penicillin/streptomycin. The skull tissue blocks are digested at 37° C. for 6 hours, with the cell tissue blocks being pipetted and blown every hour. After the digestion of the tissue blocks is complete to obtain a mixture, the mixture is filtered through a 70-μm cell strainer, centrifuged at 1000 revolutions per minute (rpm) for 5 minutes, and then resuspended in α-MEM culture medium to obtain mixed cells. The mixed cells are inoculated in a well plate to obtain osteoblasts, and the osteoblasts are cultured in complete α-MEM medium containing 10% fetal bovine serum. When a cell adhesion convergence degree of the osteoblasts reaches 60-70%, the osteoblasts are induced for osteogenic differentiation with an osteogenic culture medium containing ascorbic acid (50 g/mL) and β-glycerophosphate (10 millimoles per liter abbreviated as mM) for 7 days before the cells are collected for the next step of the experiment.

1.1.7 Extraction of Messenger Ribonucleic Acid (mRNA) of Cells and Detection of Reverse Transcription Quantitative Polymerase Chain Reaction (RT-qPCR)

TRIzol reagent (i.e., TRI reagent®) is used to harvest and extract total RNA from the osteoblast cultures. The mRNA is then reverse transcribed into complementary deoxyribonucleic acid (DNA) using the TaqMan reverse transcription kit (Biomake). After that, a real-time quantitative polymerase chain reaction (RT-qPCR) is conducted with the SYBR Premix Ex Taq™ II. The primer sequences are shown in Table 1, where β-actin is utilized as the reference gene to provide a basis for quantitative analysis. The RT-qPCR gene primer sequences are as indicated in Table 1.

TABLE 1

| Gene | Primer sequence |
|---|---|
| Ppmla | Forward: 5′-GGACAAGTACCTGGAGAGCAGA-3′ (SEQ ID NO: 1) |
| | Reverse: 5′-GGGATGTTCTCACTGGCTAACG-3′ (SEQ ID NO: 2) |
| Sp7 | Forward: 5′-AATAGTGGGCAACTGGAGGG-3′ (SEQ ID NO: 3) |
| | Reverse: 5′-GAACAGAGCAGGCAGGTGAA-3′ (SEQ ID NO: 4) |
| Bglap | Forward: 5-TCTGACAAACCTTCATGTCC-3′ (SEQ ID NO: 5) |
| | Reverse: 5′-AAATAGTGATACCGTAGATGCG-3′ (SEQ ID NO: 6) |
| Alpl | Forward: 5′-TGACCTTCTCTCCTCCATCC-3′ (SEQ ID NO: 7) |
| | Reverse: 5′-CTTCCTGGGAGTCTCATCCT-3′ (SEQ ID NO: 8) |
| β-actin | Forward: 5′-GGAGATTACTGCCCTGGCTCCTA-3′ (SEQ ID NO: 9) |
| | Reverse: 5′-GACTCATCGTACTCCTGCTTGCTG-3′ (SEQ ID NO: 10) |

### 1.2 Results

### 1.2.1 Effects of Miltefosine on Proliferation and Differentiation of Osteoblasts In Vitro

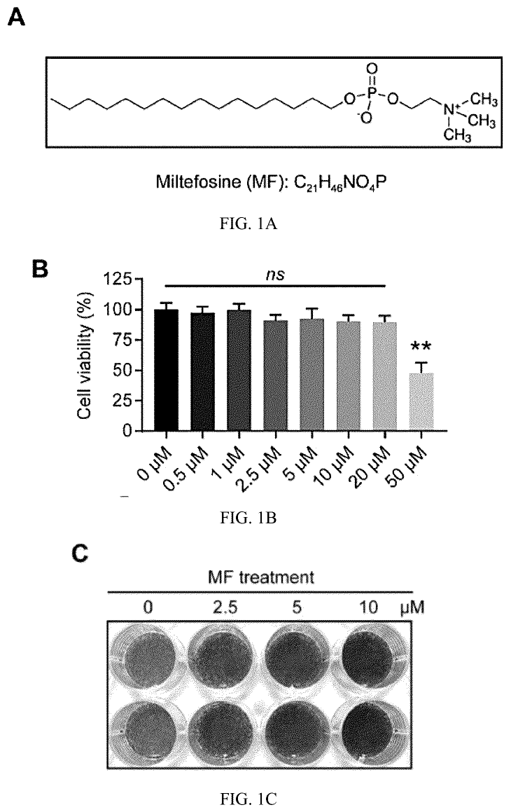

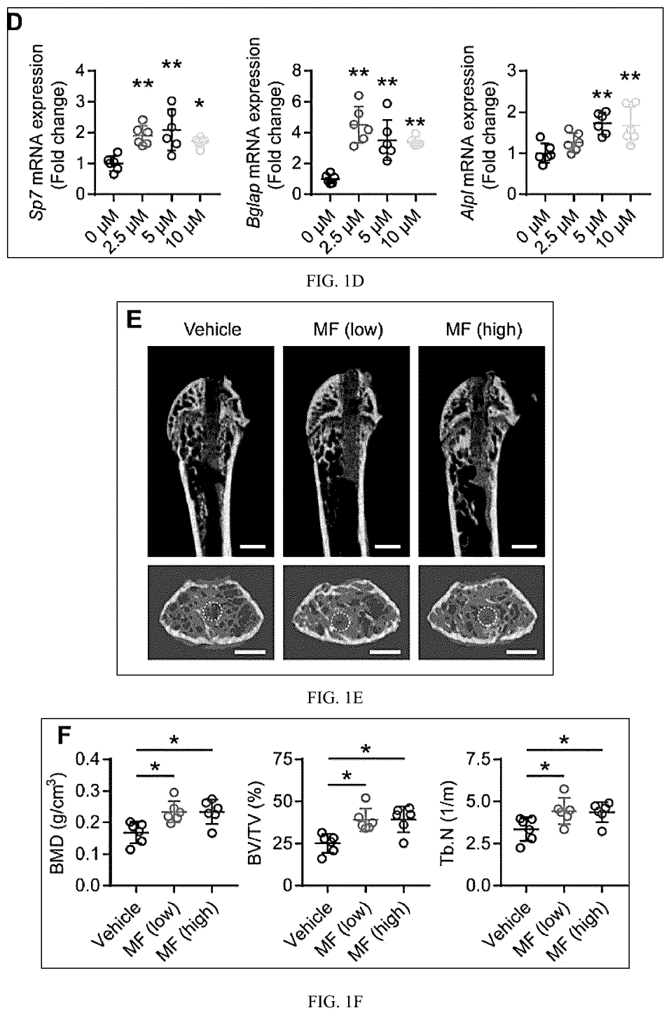

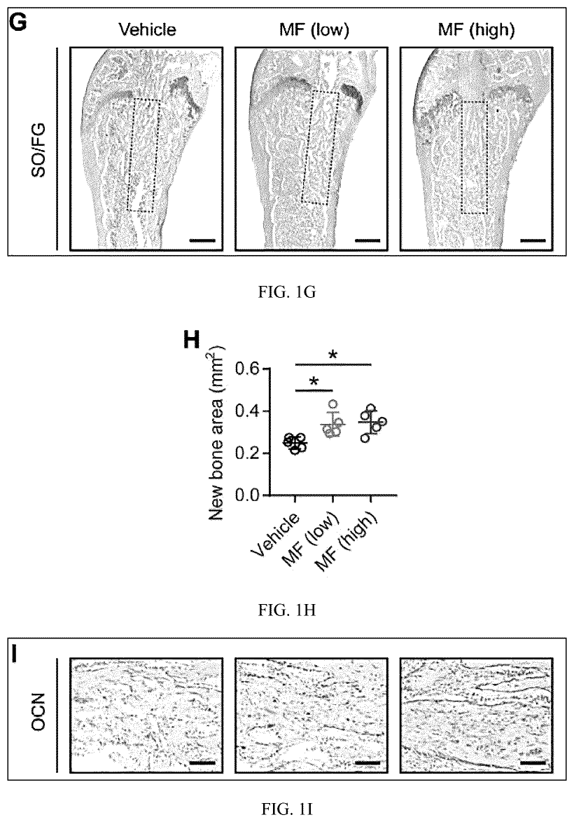

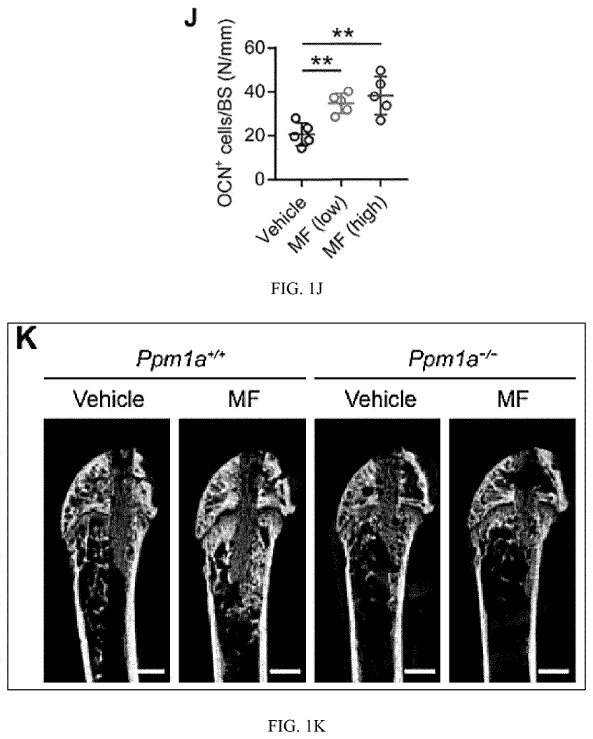

The chemical formula of the miltefosine is C₂₁H₄₆NO₄P (as shown in FIG. 1A). The cell counting kit-8 (CCK-8) assay shows that the miltefosine at concentrations ranging from 0.5 micromoles per liter (μM) to 20 μM in vitro has no significant effect on the viability of the primary osteoblasts, while a concentration of 50 μM causes approximately 50% cell death of the primary osteoblasts (as shown in FIG. 1B). Subsequent in vitro studies use drug concentrations of 2.5 μM, 5.0 μM, and 10 μM. Alkaline phosphatase staining results suggest that the intervention of the miltefosine in vitro can significantly enhance the osteogenic differentiation capacity of osteoblasts (as shown in FIG. 1C). At the same time, RT-PCR results indicate that the miltefosine can upregulate the expression of osteogenic genes Sp7, Bglap, and Alpl, promoting bone formation (as shown in FIG. 1D). In summary, the intervention of the miltefosine in vitro can effectively promote the osteogenic differentiation of the osteoblasts.

### 1.2.2 Effects of Miltefosine on Bone Defect Repair in Mice

By constructing a mouse model with femoral distal bone defect and intervening with the miltefosine, the effects in vivo of the intervention on bone repair are observed. μ-CT scans of femur sample from the mouse model one week after the mouse model is created show that the intervention of the miltefosine can effectively accelerate bone repair (as shown in FIG. 1E). Further CT analysis results indicate that the miltefosine can increase the BMD, BV/TV, and trabecular number (Tb.N) in the bone defect repair area (as shown in FIG. 1F). At the same time, SO/FG staining confirms that the area of new bone in the bone defect area of the mouse model with the intervention of the miltefosine is significantly larger than that of mouse in the control (vehicle) group (FIGS. 1G and 1H). Meanwhile, compared to the control group, an expression level of osteocalcin (OCN) in the bone defect repair area of the group intervened with the miltefosine is significantly increased (FIGS. 1I and 1J). Importantly, the miltefosine could not exert the above osteogenic effects in Ppm1a knockout (Ppm1a^{-/-}) mice (FIG. 1K), a phenomenon that is also consistent with the analysis results of μ-CT (FIG. 1L). Therefore, these data suggest that intervention in vivo with the miltefosine can effectively accelerate the repair of bone defects in mouse, and this effect is dependent on PPM1A.

### 1.3 Conclusion

### 1.3.1 the Miltefosine can Promote Osteogenic Differentiation of Osteoblasts In Vitro.

### 1.3.2 the Miltefosine can Accelerate the Repair of Bone Defects in Mouse.

### Embodiment 2

A study on the effect with intervention of miltefosine on postmenopausal osteoporosis

### 2.1 Method

### 2.1.1 Constructing an Ovariectomy (OVX) Model Mouse

All mice are subjected to perform bilateral ovariotomy (destabilization of medial meniscus, OVX) to construct an estrogen-deficient osteoporosis model. Twelve-week-old mice are anesthetized by intraperitoneal injection of 2% isoflurane gas, and after the adequate anesthesia, the dorsal and lumbar areas of the twelve-week-old mice are shaved. The skin and peritoneum of the dorsal and lumbar areas are then incised, followed by ligation of the bilateral fallopian tubes and removal of the bilateral ovaries. In a sham-operated group, the ovaries and fallopian tubes are kept intact, with only the skin and peritoneum being incised, followed by disinfection and skin suture.

### 2.1.2 Animal Sample Processing and Section Preparation

All mice are euthanized with CO₂ at eight weeks of post-surgery. Bilateral femur samples are collected and fixed at room temperature in 4% PFA for 3 days. Subsequently, the bilateral femur samples are decalcified in a 14% EDTA solution for 2 weeks, with the decalcification solution being changed every other day. After the decalcification, the decalcified bilateral femur samples are dehydrated using an automatic dehydrator. All samples are embedded in paraffin to obtain paraffin blocks and then sectioned to obtain tissue sample sections, with a section thickness of 4 m.

### 2.1.3 Imaging Observation

All the tissue samples are scanned using a high-resolution μ-CT scanner with scanning parameters set at 45 kV, 500μA, and an exposure time of 780 ms. The distal metaphysis of the femur is selected as ROI for analysis. Quantitative analysis of bone tissue microstructural parameters is performed on the ROI area, including BMD, structural model index (SMI), connectivity density (Conn.Dn), BV/TV, Tb.N, trabecular thickness (Tb.Th), and trabecular separation (Tb.Sp).

### 2.1.4 Observation of Organizational Morphology

Before staining, the tissue sample sections are placed in an oven at 60° C. for 4 hours to enhance the adhesion of the tissues to the slides. After the enhancing, the tissue sample sections are taken out to stand for 15 minutes to return to room temperature. The tissue sample sections are then deparaffinized and rehydrated using xylene and a graded series of alcohol. Subsequently, the tissue sample sections are stained using an alcian blue hematoxylin/orange G (ABH/OG) staining. A specific staining steps are as follows: firstly, the tissue sample sections are differentiated in 1% hydrochloric acid-alcohol for 30 seconds, then stained in an alcian blue hematoxylin solution for 1 hour. After the staining, the tissue sample sections are rinsed with distilled water three times, each for 3 minutes. Subsequently, the tissue sample sections are differentiated again in 1% hydrochloric acid-alcohol for 3 seconds, followed by rinsing with distilled water three times, each for 1 minute. After that, the tissue sample sections are placed in a 0.5% ammonia solution for 15 seconds to perform the counterstaining to blue. Then, the tissue sample sections are placed in a 95% alcohol solution for 1 minute, and transferred to an eosin/orange G working fluid for staining for 1 minute and 30 seconds. Finally, the tissue sample sections are dehydrated through a graded series of alcohol and cleared with xylene, and the tissue sample sections are sealed with a mounting medium.

### 2.1.5 Immunohistochemical Analysis

The tissue sample sections are taken to place in an oven at 60° C. to bake overnight. The next day, the tissue sample sections are taken out to stand for 15 minutes to return to room temperature, then the tissue sample sections are deparaffinized and rehydrated using xylene and a graded series of alcohol. A heat retrieval method (also referred to as heat fixation method) is employed by immersing the tissue sample sections in a sodium citrate buffer solution and placing the tissue sample sections in the sodium citrate buffer solution in an oven at 60° C. for 4 hours to complete antigen retrieval, thereby exposing antigen epitopes fully. The tissue sample sections are treated with a 0.3% Triton X-100 solution for permeabilization, then 100 μL of an endogenous peroxidase blocker are added into each tissue sample section to incubate at room temperature for 10 minutes. After the incubating, 100 μL of a normal goat serum working solution are added into each issue sample section for blocking, followed by incubating at room temperature for 15 minutes, and then the normal goat serum in each issue sample section is discarded. After the discarding, 100 μL of a primary antibody reagent are added into each issue sample section to incubate overnight at 4° C. The next day, the issue sample sections are taken out to return to room temperature, the primary antibody reagent in each issue sample section is washed off with PBS buffer, and then 100 μL of biotinylated goat anti-rabbit IgG polymer, 100 μL of horseradish peroxidase-labeled streptavidin working solution are sequentially added into each issue sample section, and then an appropriate amount of freshly prepared DAB solution is added for a color development. After the color development is complete, the tissue sample sections are placed in a hematoxylin staining solution for 20 seconds and differentiated with 1% hydrochloric acid alcohol, and followed by rinsing with tap water to blue. Finally, the tissue sample sections are dehydrated through a graded series of alcohol and cleared with xylene, and the tissue sample sections are sealed with neutral resin.

### 2.2 Results

2.2.1 Intervention with Miltefosine can Improve Bone Loss in Postmenopausal Mice

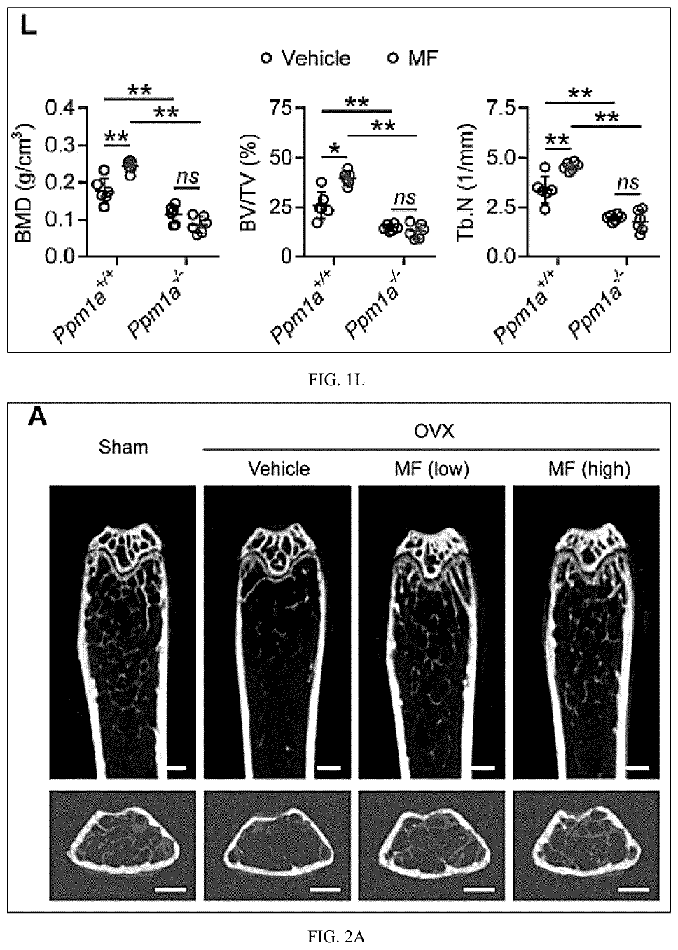

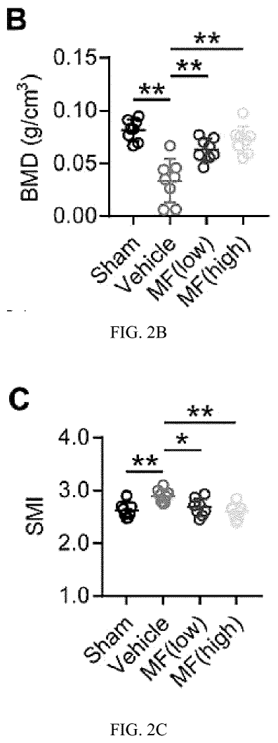

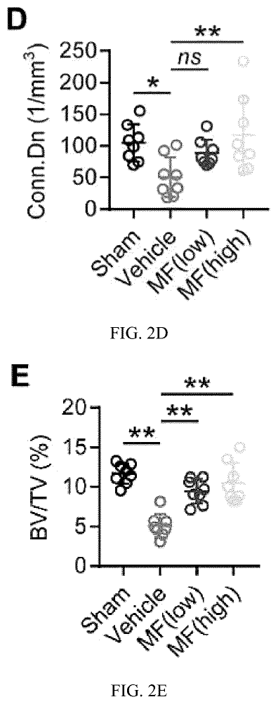

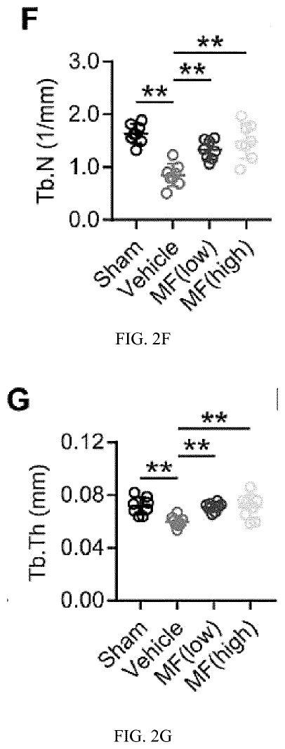

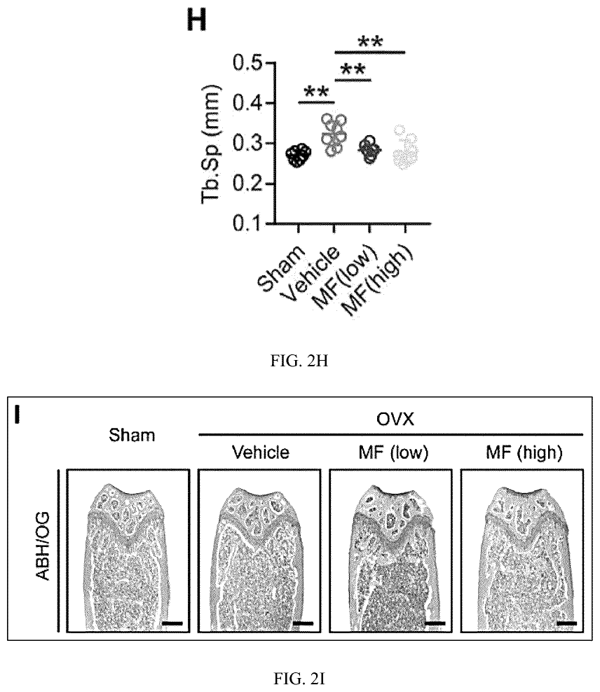

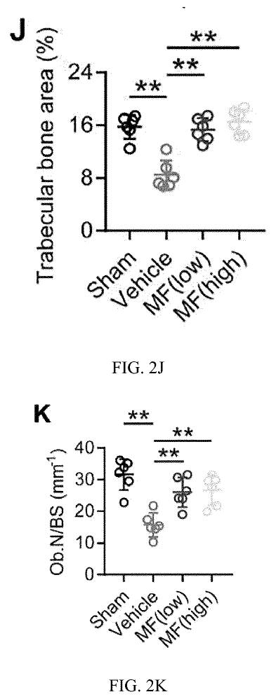

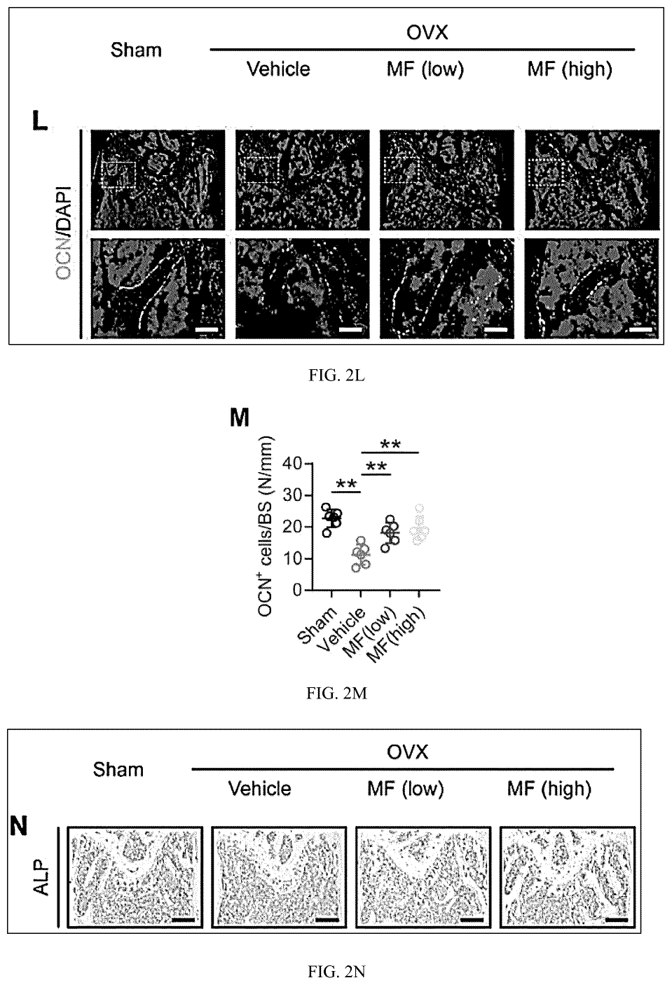

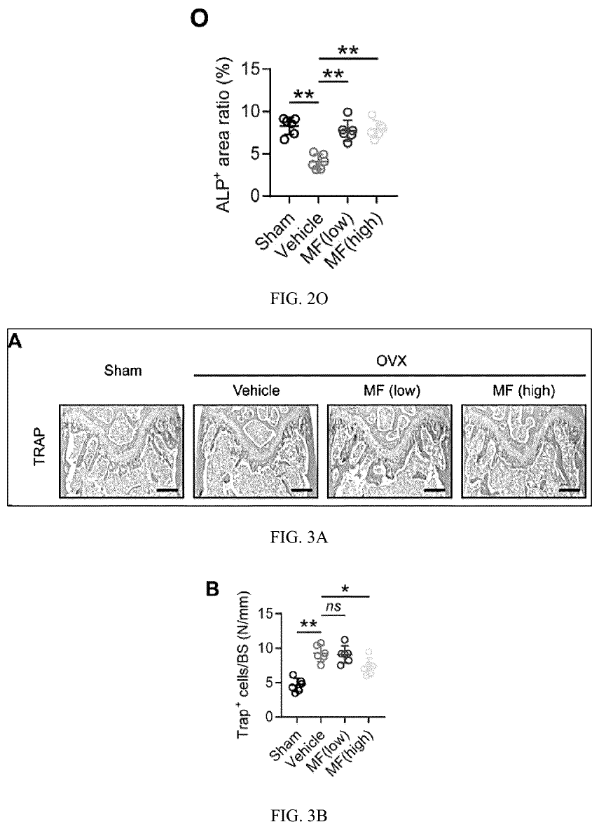

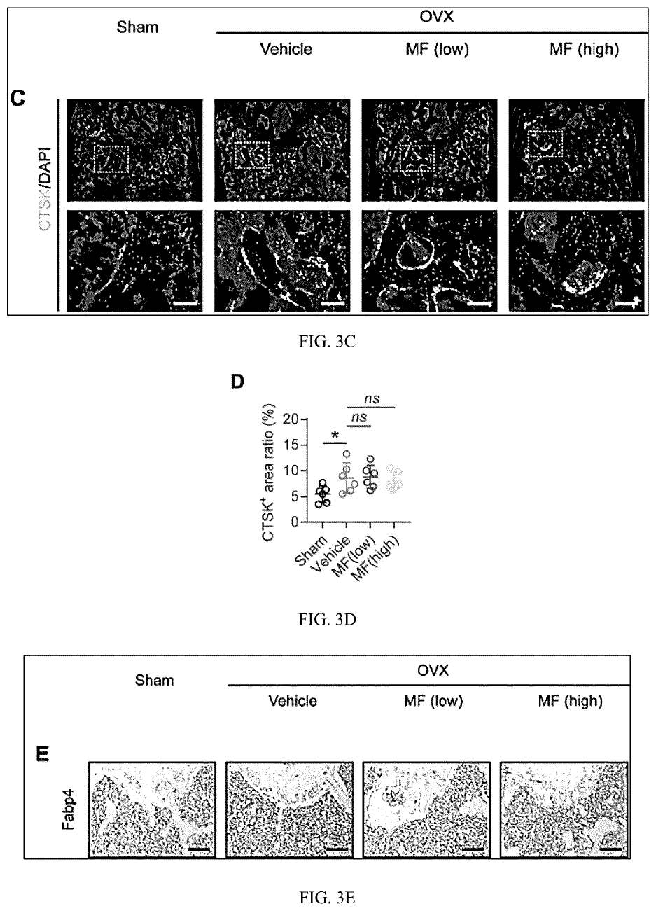

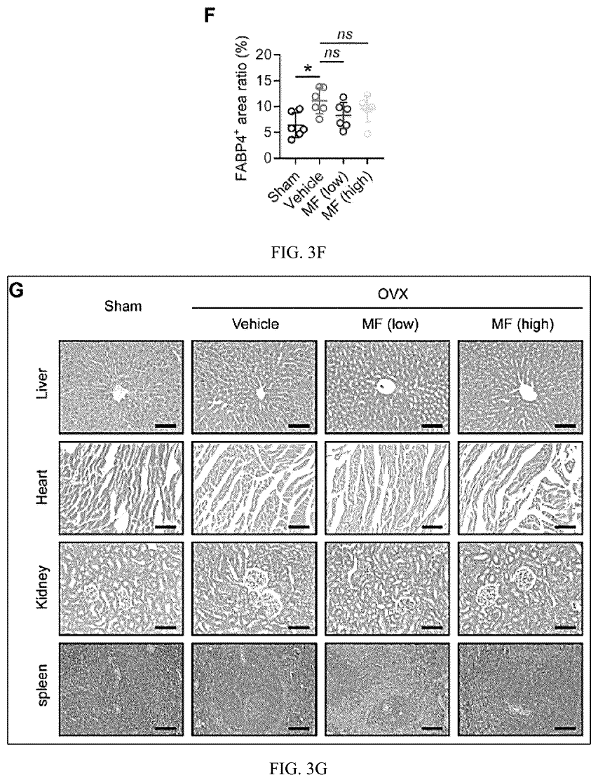

The micro-CT scanning reveals that after 8 weeks post-ovariectomy (OVX), the bone mass at the distal metaphysis of the femur in the model group mice is significantly reduced, while the bone loss in the low-dose and high-dose miltefosine-treated groups is significantly less than that in the control group with sham-operated (FIG. 2A). Further microstructural analysis indicates that the intervention with miltefosine can significantly increase the BMD at the distal femur (FIG. 2B) and improve SMI of the bone tissue (FIG. 2C) and Conn.Dn (FIG. 2D). At the same time, analysis of the trabecular bone shows that the BV/TV, Tb.N and Tb.Th in miltefosine-treated mice are all significantly increased compared to the control group, while the Tb.Sp is markedly reduced (FIGS. 2E-2H). Histomorphometric analysis also shows that the intervention with miltefosine can reduce bone loss at the distal femur (FIGS. 2I-2J) and increase the number of osteoblasts on the trabecular bone surface (FIG. 2K). In addition, immunofluorescence staining for OCN and alkaline phosphatase (ALP) immunohistochemical staining both show that the miltefosine can significantly promote the bone formation of OVX mice (FIGS. 2L-20). The TRAP staining of the distal femur sections shows that the intervention with low-dose miltefosine does not affect the number of TRAP-positive cells in OVX mice, while the intervention with high-dose miltefosine slightly reduces the number of TRAP-positive cells (FIGS. 3A-3B), and the expression level of the mature osteoclast marker of cathepsin K (CTSK) shows no significant difference between the control group and the miltefosine-treated groups (FIGD. 3C-3D). Meanwhile, fatty acid binding protein 4 (Fabp4) immunohistochemical staining suggests that the miltefosine has no obvious effect on the formation of adipocytes (FIGS. 3E-3F). The results indicate that the intervention with miltefosine can effectively reduce bone loss in postmenopausal mice and improve their bone microstructure, and this effect is mainly achieved by enhancing bone formation.

### 2.2.2 Biosafety Evaluation of Miltefosine

The livers, hearts, kidneys, and spleens of the mice in the OVX model post-intervention with miltefosine for 8 weeks are collected, and the tissue morphology of these organs is observed through hematoxylin and eosin (HE) staining. The integrity, tissue structure, and morphology of the main organs in groups with the interventions of low-dose and high-dose miltefosine are similar to those in the control group mice, with no obvious lesions or pathological changes observed (FIG. 3G). This suggests that 8 weeks of the intervention with miltefosine has no significant toxic side effects on the livers, hearts, kidneys, and spleens of the OVX model mice, indicating that the intervention of miltefosine for 8 weeks is bio-safe for the OVX model mice.

### CONCLUSION

2.3.1 Intervention with Miltefosine can Promote the Osteogenic Function of Postmenopausal Mice and Effectively Improve Bone Loss.
2.3.2 Intervention with Miltefosine has Biosafety in Postmenopausal Mice.

Those skilled in the art will easily come up with other implementation solutions of the disclosure after considering the content disclosed in the specification and practice. The disclosure aims to cover any variations, uses, or adaptive changes of the disclosure, which follow the general principles of the disclosure and include common knowledge or customary technical means in the related art not disclosed in the disclosure.

It should be understood that the disclosure is not limited to the precise structure described above and shown in the drawings, and various modifications and changes can be made without departing from its scope.

What is claimed is:

1. An application method of miltefosine, comprising:
promoting bone formation and preventing and treating osteoporosis by using the miltefosine, wherein the miltefosine is hexadecylphosphocholine (C₂₁H₄₆NO₄P) shown in a formula I, and the formula I is expressed as follows: 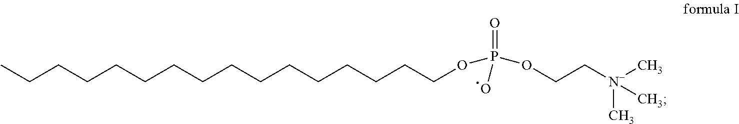
further comprising:
using the miltefosine to enhance osteogenic differentiation of osteoblasts;
using the miltefosine to increase a bone density of newly formed bone in a repair area;
using the miltefosine to increase a bone volume fraction of the newly formed bone in the repair area;
using the miltefosine to enhance a trabecular bone of the newly formed bone in the repair area;
using the miltefosine to increase a bone density in a distal femur of a postmenopausal women;
using the miltefosine to reduce a trabecular separation in the distal femur of the postmenopausal women; and
using the miltefosine to reduce bone loss in the distal femur of the postmenopausal women and to increase a number of the osteoblasts on a surface of the trabecular bone.

2. The application method as claimed in claim 1, wherein the miltefosine is an osteogenic agent and is in a form of a solid powder.

3. The application method as claimed in claim 1, wherein the miltefosine is a medication catalyzing an activity of protein phosphatase magnesium-dependent 1A (PPM1A) in the osteoblasts.

4. The application method as claimed in claim 1, wherein the osteoporosis comprises postmenopausal osteoporosis.

5. The application method as claimed in claim 1, wherein an effective dose of the miltefosine for promoting the bone formation is in a range of 5-10 milligrams per kilogram (mg/kg) body weight.
